(19) United States
(12) Patent Application Publication
(10) Pub. No.: US 2020/0107726 A1
(43) Pub. Date: Apr. 9, 2020

(54) SUCTION INSTRUMENT WITH DISSECTING TIP AND AXIALLY OFFSET SENSORS

(71) Applicant: Acclarent, Inc., Irvine, CA (US)

(72) Inventors: Henry F. Salazar, Pico Rivera, CA (US); Jordan R. Trott, Redondo Beach, CA (US); Jetmir Palushi, Irvine, CA (US); Athanasios Papadakis, Newport Beach, CA (US); Fatemeh Akbarian, Rancho Palos Verdes, CA (US)

(21) Appl. No.: 16/553,392

(22) Filed: Aug. 28, 2019

Related U.S. Application Data
(60) Provisional application No. 62/741,609, filed on Oct. 5, 2018.

(51) Int. Cl. A61B 5/00; A61B 5/06; A61B 17/32

(52) U.S. Cl. CPC A61B 5/0055; A61B 1/227; A61B 17/320016; A61B 5/065

(57) ABSTRACT

An apparatus includes a shaft assembly, a distal tip member, a dissection member, and a position sensor. The distal tip member has an opening in fluid communication with a lumen of the shaft assembly. A longitudinal axis extends centrally through the opening and lumen at the distal tip member. The dissection member extends distally from the distal tip member. The first position sensor is operable to generate signals indicating a position of the distal tip member in space.

### PRIORITY

This application claims priority to U.S. Provisional patent application Ser. No. 62/741,609, entitled “Suction Instrument with Dissecting Tip and Axially Offset Sensors,” filed Oct. 5, 2018, the disclosure of which is incorporated by reference herein.

### BACKGROUND

In some instances, it may be desirable to operate within or adjacent to an anatomical passageway of a patient, such as performing an incision of mucosa, removal of bone, or dilation of an anatomical passageway. Such operations may occur within anatomical passageways such as ostia of paranasal sinuses (e.g., to treat sinusitis), the larynx, the Eustachian tube, or other passageways within the ear, nose, or throat, etc. In addition to the above described operations, or similar operations, it may be desirable to apply suction and/or irrigation within or adjacent to an anatomical passageway before, during, or after the above described operations, or similar operations. One method of applying suction within or adjacent to an anatomical passageway of a patient involves obtaining a suction device having an elongate shaft defining a lumen terminating at an open distal end of the elongated shaft, where the lumen is in fluid communication with an external suction source. An operator may then insert the distal end of the elongate shaft within the nostril or mouth of a patient toward a desired location within the patient. With the distal end of the elongate shaft inserted within the patient, an operator may manipulate the suction device and/or suction source in order to remove extraneous and/or undesired matter near or within an anatomical passageway of a patient. Applying suction and/or irrigation during an operation may be beneficial for multiple purposes as will be apparent to those skilled in the art.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation systems that may be used in IGS procedures is the CARTO® 3 System by Biosense-Webster, Inc., of Irvine, Calif. In some IGS procedures, a digital tomographic scan (e.g., CT or MM, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

While several systems and methods have been made and used in surgical procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic view of an exemplary surgery navigation system being used on a patient seated in an exemplary medical procedure chair;

FIG. 2 depicts a perspective view of an exemplary suction instrument that may be used with the surgery navigation system of FIG. 1;

FIG. 3 depicts an exploded view of a distal portion of the suction instrument of FIG. 2; and

FIG. 4 depicts an enlarged perspective view of the distal end of an exemplary variation of the suction instrument of FIG. 2.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

### I. EXEMPLARY IMAGE GUIDED SURGERY NAVIGATION SYSTEM

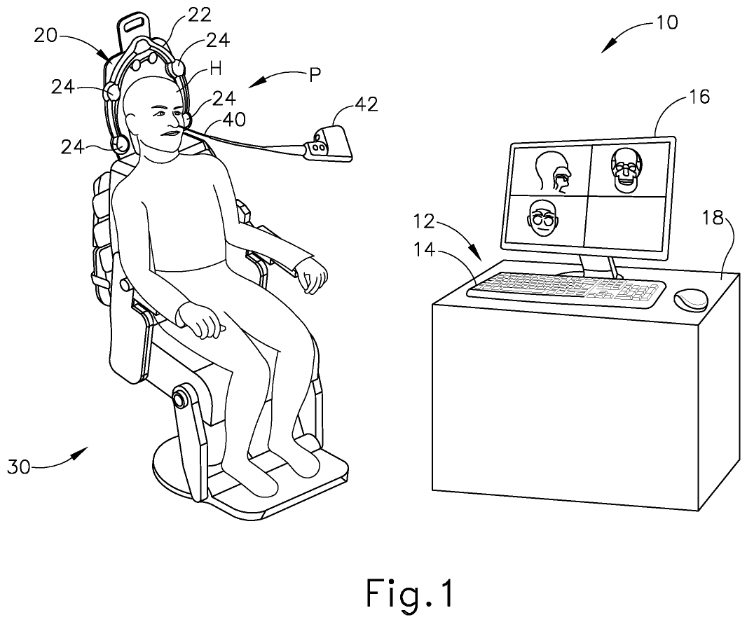

When performing a medical procedure within a head (H) of a patient (P), it may be desirable to have information regarding the position of an instrument within the head (H) of the patient (P), particularly when the instrument is in a location where it is difficult or impossible to obtain an endoscopic view of a working element of the instrument within the head (H) of the patient (P). FIG. 1 shows an exemplary IGS navigation system (10) enabling an ENT procedure to be performed using image guidance. In addition to or in lieu of having the components and operability described herein IGS navigation system (10) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; and U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein.

IGS navigation system (10) of the present example comprises a field generator assembly (20), which comprises set of magnetic field generators (24) that are integrated into a horseshoe-shaped frame (22). Field generators (24) are operable to generate alternating magnetic fields of different frequencies around the head (H) of the patient (P). A navigation guidewire (40) is inserted into the head (H) of the patient (P) in this example. Navigation guidewire (40) may be a standalone device or may be positioned on an end effector or other location of a medical instrument such as a surgical cutting instrument or dilation instrument. In the present example, frame (22) is mounted to a chair (30), with the patient (P) being seated in the chair (30) such that frame (22) is located adjacent to the head (H) of the patient (P). By way of example only, chair (30) and/or field generator assembly (20) may be configured and operable in accordance with at least some of the teachings of U.S. patent application Ser. No. 15/933,737, entitled “Apparatus to Secure Field Generating Device to Chair,” filed Mar. 23, 2018, the disclosure of which is incorporated by reference herein.

IGS navigation system (10) of the present example further comprises a processor (12), which controls field generators (24) and other elements of IGS navigation system (10). For instance, processor (12) is operable to drive field generators (24) to generate alternating electromagnetic fields; and process signals from navigation guidewire (40) to determine the location of a sensor in navigation guidewire (40) within the head (H) of the patient (P). Processor (12) comprises a processing unit communicating with one or more memories. Processor (12) of the present example is mounted in a console (18), which comprises operating controls (14) that include a keypad and/or a pointing device such as a mouse or trackball. A physician uses operating controls (14) to interact with processor (12) while performing the surgical procedure.

Navigation guidewire (40) includes a sensor (not shown) that is responsive to positioning within the alternating magnetic fields generated by field generators (24). A coupling unit (42) is secured to the proximal end of navigation guidewire (40) and is configured to provide communication of data and other signals between console (18) and navigation guidewire (40). Coupling unit (42) may provide wired or wireless communication of data and other signals.

In the present example, the sensor of navigation guidewire (40) comprises at least one coil at the distal end of navigation guidewire (40). When such a coil is positioned within an alternating electromagnetic field generated by field generators (24), the alternating magnetic field may generate electrical current in the coil, and this electrical current may be communicated along the electrical conduit(s) in navigation guidewire (40) and further to processor (12) via coupling unit (42). This phenomenon may enable IGS navigation system (10) to determine the location of the distal end of navigation guidewire (40) or other medical instrument (e.g., dilation instrument, surgical cutting instrument, etc.) within a three-dimensional space (i.e., within the head (H) of the patient (P), etc.). To accomplish this, processor (12) executes an algorithm to calculate location coordinates of the distal end of navigation guidewire (40) from the position related signals of the coil(s) in navigation guidewire (40). While the position sensor is located in guidewire (40) in this example, such a position sensor may be integrated into various other kinds of instruments, including those described in greater detail below.

Processor (12) uses software stored in a memory of processor (12) to calibrate and operate IGS navigation system (10). Such operation includes driving field generators (24), processing data from navigation guidewire (40), processing data from operating controls (14), and driving display screen (16). In some implementations, operation may also include monitoring and enforcement of one or more safety features or functions of IGS navigation system (10). Processor (12) is further operable to provide video in real time via display screen (16), showing the position of the distal end of navigation guidewire (40) in relation to a video camera image of the patient's head (H), a CT scan image of the patient's head (H), and/or a computer generated three-dimensional model of the anatomy within and adjacent to the patient's nasal cavity. Display screen (16) may display such images simultaneously and/or superimposed on each other during the surgical procedure. Such displayed images may also include graphical representations of instruments that are inserted in the patient's head (H), such as navigation guidewire (40), such that the operator may view the virtual rendering of the instrument at its actual location in real time. By way of example only, display screen (16) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (16).

The images provided through display screen (16) may help guide the operator in maneuvering and otherwise manipulating instruments within the patient's head (H) when such instruments incorporate navigation guidewire (40). It should also be understood that other components of a surgical instrument and other kinds of surgical instruments, as described below, may incorporate a sensor like the sensor of navigation guidewire (40).

### II. EXEMPLARY SUCTION INSTRUMENT WITH DISSECTION FEATURE

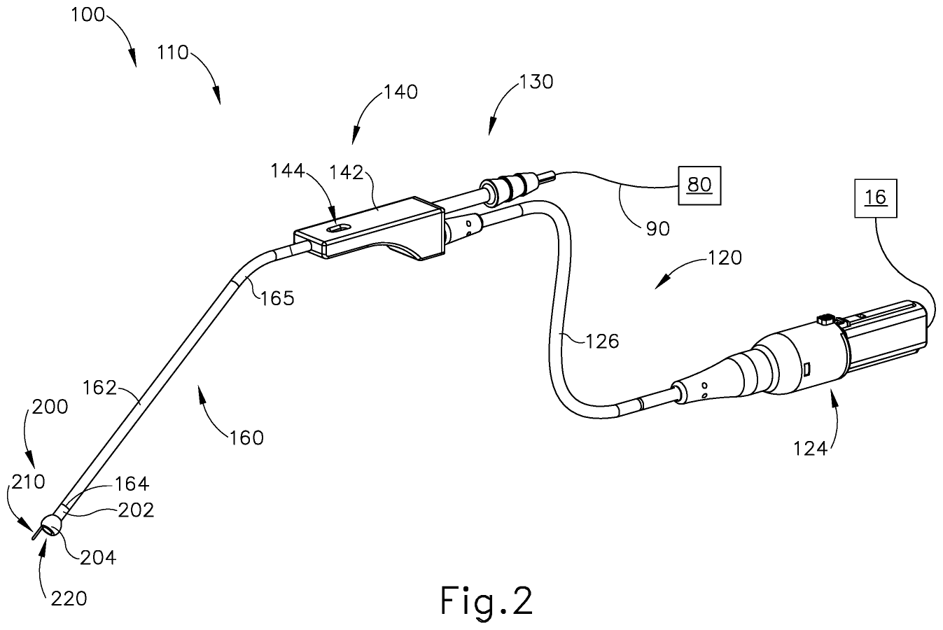

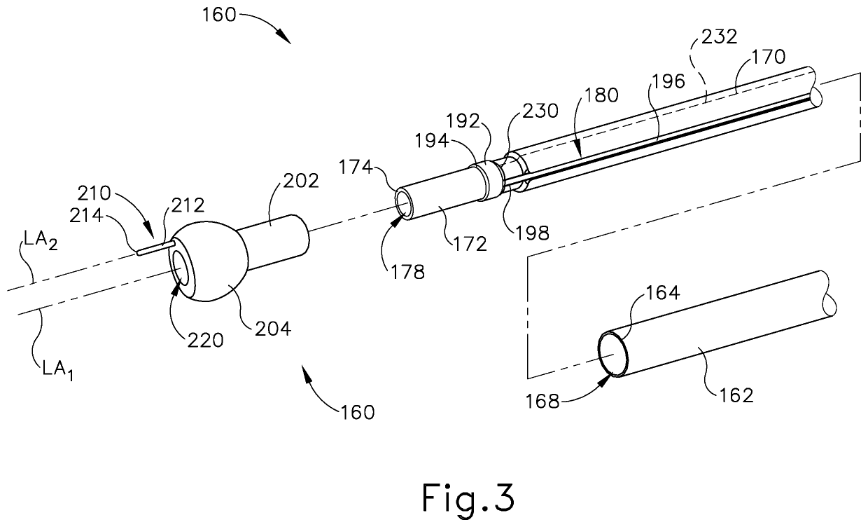

Various surgical procedures may warrant the use of a suction instrument in order to clear fluids and/or debris from the surgical field and/or from other sites within a patient. For instance, suction may be desirable in FESS procedures, sinuplasty procedures, and/or in various other ENT procedures. It may also be desirable to provide image guided navigation capabilities to a suction instrument, enabling use of the suction instrument with IGS navigation system (10). Utilizing IGS navigation system (10) in conjunction with a suction may allow the operator to provide better placement and tracking of the suction instrument within the patient (P). In addition to the foregoing, it may be desirable to include a feature on a suction instrument that facilitates use of the suction instrument to perform blunt dissections or elevations of anatomical structures, particularly under guidance of IGS navigation system (10). FIGS. 2-3 show an exemplary suction instrument assembly (100) that may be used to provides these functionalities.

Instrument assembly (100) of the present example includes a suction instrument (110), console (16) of IGS navigation system (10), and a suction source (80). Suction instrument (110) is fluidly coupled with suction source (80) via a conduit (90). Suction source (80) may comprise a vacuum pump and a fluid reservoir, among other components, as is known in the art. Suction source (80) is configured to provide enough suction to pull excess fluid and/or debris through suction instrument (110).

Suction instrument (110) is in communication with IGS navigation system (10) via console (18). As will be described in greater detail below, suction instrument (110) is configured to communicate with console (18) such that processor (12) may execute an algorithm to calculate location coordinates of a selected portion of suction instrument (110). Therefore, suction instrument (110) is in communication with IGS navigation system (10) such that IGS navigation system (10) may calculate, track, and display the spatial location of a portion of suction instrument (110) relative to a three-dimensional model of the anatomy within or adjacent to a patient's nasal cavity.

Suction instrument (110) of this example comprises a coupling unit (120), a proximal suction conduit port (130), a grip portion (140), and an elongate cannula assembly (160). A distal end of cannula assembly (160) may be inserted, transnasally or otherwise, within or adjacent to a nasal cavity of a patient (or elsewhere within a patient) to provide suction. As will be described in greater detail below, cannula assembly (160) includes a set of position sensors (192, 230) that may communicate data corresponding to the 3-dimensional spatial position of cannula assembly (160) to console (18) via coupling unit (120).

Coupling unit (120) includes a sensor coupling (122), a console coupling (124), and a cable (126) connecting grip portion (140) with a console plug (124). Console plug (124) is configured to plug into a corresponding socket (not shown) of console (18), to thereby establish a path for communication between suction instrument (110) and console (18) via cable (126). In some other versions, suction instrument (110) is in wireless communication with console (18). Various suitable ways in which wireless communication may be provided between suction instrument (110) and console (18) will be apparent to those skilled in the art in view of the teachings herein.

Proximal suction conduit port (130) includes a proximal barbed fitting that couples conduit (90) with a hollow interior region of body (142) of grip portion (140). This hollow interior region provides a pathway for fluid communication between conduit (90) and cannula assembly (160), thereby providing suction from suction source (80) to distal end (200) of cannula assembly (160). Body (142) may be grasped by an operator such that the operator may manipulate and control suction instrument (110). Body (142) further includes a vent opening (144) that is in fluid communication with the hollow interior of body (142). An operator may control the suction communicated from suction source (80) to cannula assembly (160) by selectively covering vent opening (144) with the thumb or other finger of the hand that is grasping grip portion (140). When vent opening (144) is covered, suction will be communicated from suction source (80) and conduit (90) to cannula assembly (160). When vent opening (144) is uncovered, suction from suction source (80) and conduit (90) will be communicated to atmosphere via vent opening (144).

Elongate cannula assembly (160) of the present example includes external sheath (162), an interior suction tube (170), a pair of position sensors (192, 230), and a distal cap (202). As will be described in greater detail below, external sheath (162), interior suction tube (170), and distal cap (202) are configured to cooperatively house position sensors (192, 230) such that position sensors (192, 230) are spatially fixed relative to the rest of elongate cannula assembly (160). As will also be described in greater detail below, each position sensor (192, 230) is configured to generate an electrical current in response to an alternating electromagnetic field generated by field generators (24); and then communicate that electrical current to console (18) via to coupling unit (120) such that IGS navigation system (10) may determine the location of the distal end (200) of elongate cannula assembly (160) within the head (H) of the patient (P).

External sheath (162) has an open distal end (164). External sheath (162) also defines a hollow interior (168) that extends from open proximal end (166) to open distal end (164). Hollow interior (168) is dimensioned to house a portion interior suction tube (170) as well as a portion of a communication cable (196) extending within and along a guided path (180) defined by interior suction tube (170). Communication cable (196) may include one or more wires or other electrically conductive features. The portion of communication cable (196) extending along cannula assembly (160) may be housed between guide path (180) and external sheath (162). While in the current example, guide path (180) of internal suction tube (170) houses communication cable (196) external to grip portion (140), it should be understood that any other suitable channel/combination of tubes may be used to house communication cable (196). For instance, external sheath (162) may be omitted, while internal suction tube (170) may define a lumen fluidly isolated from suction lumen (178) configured to entirely house communication cable (196). Any other suitable housing configuration may be used as would be apparent to those skilled in the art in view of the teachings herein.

Interior suction tube (170) also includes a narrowed distal portion (172) that extends distally relative to open distal end (164) of external sheath (162) when properly assembled. As will be described in greater detail below, narrowed distal portion (172) is dimensioned to coaxially receive position sensor (192) such that position sensor (192) is fixed relative to narrowed distal portion (172). Position sensor (230) is laterally offset from narrowed distal portion (172). Narrowed distal portion (172) is also dimensioned to receive distal cap (202) such that distal cap (202) covers narrowed distal portion (172) and position sensors (192, 230).

Interior suction tube (170) defines a guided path (180) extending proximally from the proximal end of narrowed distal portion (172). As mentioned above, and as will be described below, guided path (180) is dimensioned to house communication cable (196) cooperatively with external sheath (162). Interior suction tube (170) also defines suction lumen (178) that extends proximally from open distal end (174) to grip portion (140), such that suction lumen (178) of suction tube (170) may receive suction from conduit (90) and suction source (80). Therefore, when a distal end (200) of elongate cannula assembly (160) is inserted within or adjacent to a nasal cavity or other desired location of a patient, elongate cannula assembly (160) may provide suction to pull away excess fluid and/or debris away from the desired location via suction lumen (178), in accordance with the teachings herein.

While suction lumen (178) has a circular cross-sectional profile in the present example, it should be understood that suction lumen (178) may instead have an elliptical cross-sectional profile or some other non-circular cross-sectional profile, if desired. A non-circular cross-sectional profile may provide additional clearance for other instruments to be positioned simultaneously in the same anatomical passageway (e.g., nasal cavity) with cannula assembly (160).

Cannula assembly (160) has an open distal end (200) and a bent region (165) formed just distal to grip portion (140). Bent region (165) defines a bend angle that is selected to facilitate insertion of distal end (200) in a patient by an operator grasping grip portion (140). Various suitable bend angles that may be used will be apparent to those of ordinary skill in the art in view of the teachings herein. In the present example, cannula assembly (160) is rigid such that cannula assembly (160) maintains the bend of bent region (165) and does not buckle during insertion into a patient's nasal cavity. By way of example only, external sheath (162) and internal suction tube (170) may be formed of stainless steel (e.g., a stainless steel hypotube, etc.) and/or any other suitable rigid material.

As shown in FIG. 3, position sensor (192) is annular in shape and is fixedly positioned coaxially on a polyimide tube (194), which is coaxially secured about narrowed distal portion (172) of interior suction tube (170). Polyimide tube (194) may provide structural stiffness and act as an insulator to help protect annular sensor (192) from contacting narrowed portion (172) of interior suction tube (170). By way of example only, position sensor (192) may comprise a wire coil wrapped about a coil axis that is coaxial with the longitudinal axis (LA₁) of interior suction tube (170). In other words, position sensor (192) may be in the form of a single-axis sensor. As noted above, position sensor (192) is configured to generate position-indicative signals, and these signals may be communicated along communication cable (196) to cable (126), ultimately reaching console (18).

Position sensor (230) is also annular or cylindraceous in shape. By way of example only, position sensor (230) may comprise a wire coil wrapped about a coil axis that is coaxial with the longitudinal axis (LA₂) of a dissection prong (210), which will be described in greater detail below. In other words, position sensor (230) may be in the form of a single-axis sensor. Position sensor (230) is coupled with a communication wire or cable (232), which is separate from communication cable (196) in this example, and which extends along the sidewall of suction tube (170). In some other versions, position sensor (230) is coupled with communication cable (196). As noted above, position sensor (230) is configured to generate position-indicative signals, and these signals may be communicated along communication cable (232) to cable (126), ultimately reaching console (18). In the present example, position sensor (230) is located proximal to position sensor (192). Other suitable locations and arrangements for position sensor (230), and other various ways in which position sensor (230) may be fixedly secured on cannula assembly (160), will be apparent to those skilled in the art in view of the teachings herein.

As also shown in FIG. 3, distal cap (202) of the present example is configured to fit over narrowed distal portion (172) of interior suction tube (170). Distal cap (202) defines a bore (220) that is configured to receive narrowed distal portion (172) of interior suction tube (170), including both position sensors (192, 230). Distal cap (202) includes a bulbous distal portion (204). In some versions, position sensor (230) is located within bulbous distal portion (204). Distal cap (202) of the present example further includes a dissection prong (210) extending distally from bulbous distal portion (204). Dissection prong (210) includes a rigid shaft (212) with an atraumatic distal tip (214). Dissection prong (210) extends along a longitudinal axis (LA₂) that is parallel to, yet laterally offset from, the longitudinal axis (LA₁) of interior suction tube (170). While longitudinal axis (LA₂) is parallel to, yet laterally offset from, the longitudinal axis (LA₁) of interior suction tube (170), longitudinal axis (LA₂) may have any other suitable relationship with the longitudinal axis (LA₁) of interior suction tube (170). By way of example only, longitudinal axis (LA₂) may be obliquely oriented relative to longitudinal axis (LA₁).

Dissection prong (210) may be used by the operator to provide blunt dissection of anatomical structures in or near the nasal cavity. The atraumatic configuration of distal tip (214) will ensure that dissection prong (210) does not impart trauma to tissue that is being bluntly dissected. It should also be understood that dissection prong (210) may be used to elevate or otherwise move anatomical structures, without necessarily performing blunt dissection, in or near the nasal cavity. Other suitable ways in which dissection prong (210) may be used will be apparent to those skilled in the art in view of the teachings herein. As the operator maneuvers distal end (200) within the nasal cavity of the patient, position sensors (192, 230) will provide position data enabling IGS navigation system (10) to provide real-time feedback to the operator indicating the position of dissection prong (210) in the nasal cavity.

By having position sensor (230) positioned coaxially about longitudinal axis (LA₂), the combination of position sensors (192, 230) may be able to provide additional information indicating the orientation of dissection prong (210), beyond the positional information that can be provided by the combination of position sensor (192).

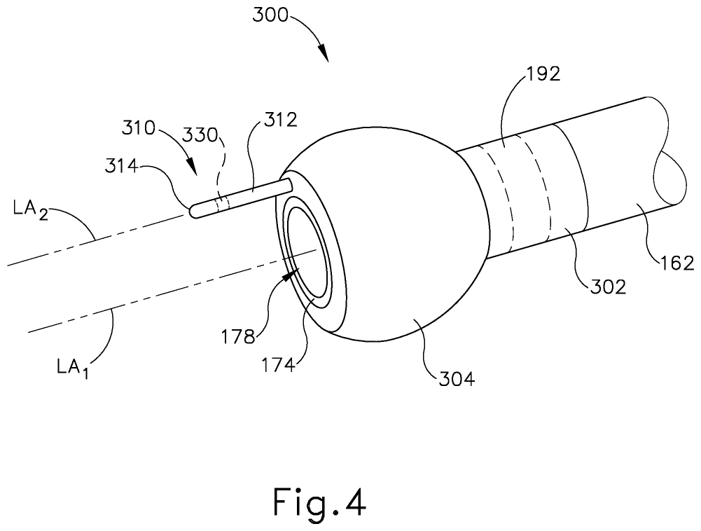

FIG. 4 shows another exemplary distal end (300) configuration that may be incorporated into cannula assembly (160). In this example, external sheath (162) and interior suction tube (170) are again used; but a different distal cap (302) is secured to narrowed distal portion (172) of interior suction tube (170). Distal cap (302) is substantially the same as distal cap (202) described above. Distal cap (302) includes a bulbous distal portion (304) and a dissection prong (210) extending distally from bulbous distal portion (204). Dissection prong (310) includes a rigid shaft (312) with an atraumatic distal tip (314). Dissection prong (310) extends along a longitudinal axis (LA₂) that is parallel to, yet laterally offset from, the longitudinal axis (LA₁) of interior suction tube (170).

Distal end (300) of this example also includes a second position sensor (330). However, unlike position sensor (230), which is positioned at a location proximal to dissection prong (310), position sensor (330) of this example is coaxially positioned about longitudinal axis (LA₂) in rigid shaft (312) of dissection prong (310). Position sensor (330) may otherwise be configured and operable like position sensors (192, 230) described above, such that position sensor (330) may generate signals indicating the position of dissection prong (310) in three-dimensional space. As the operator maneuvers distal end (300) within the nasal cavity of the patient, position sensors (192, 330) will provide position data enabling IGS navigation system (10) to provide real-time feedback to the operator indicating the position of dissection prong (310) in the nasal cavity. By having position sensor (330) in dissection prong (310), about longitudinal axis (LA₂), the combination of position sensors (192, 330) may be able to provide additional information indicating the orientation of dissection prong (310), beyond the positional information that can be provided by the combination of position sensor (192).

### III. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a shaft assembly defining a lumen; (b) a distal tip member at a distal end of the shaft assembly, the distal tip member having an opening in fluid communication with the lumen of the shaft assembly, wherein a first longitudinal axis extends centrally through the opening and lumen at the distal tip member; (c) a dissection member extending distally from the distal tip member; and (d) a first position sensor operable to generate signals indicating a position of the distal tip member in space.

### Example 2

The apparatus of Example 1, wherein the first position sensor comprises a first wire coil wrapped about a first coil axis.

### Example 3

The apparatus of Example 2, wherein the first coil axis is coaxial with the first longitudinal axis.

### Example 4

The apparatus of any one or more of Examples 1 through 3, wherein the first position sensor comprises an annular member captured radially between a distal portion of the shaft assembly and a proximal portion of the distal tip member.

### Example 5

The apparatus of Example 4, wherein the shaft assembly includes a proximal portion having a first outer diameter and the distal portion having a second outer diameter, wherein the second outer diameter is smaller than the first outer diameter, wherein the annular member is positioned on the distal portion about the second outer diameter.

### Example 6

The apparatus of any one or more of Examples 1 through 5, wherein the distal tip member has a bulbous portion.

### Example 7

The apparatus of Example 6, wherein the distal tip member further includes a cylindraceous portion located proximal to the bulbous portion.

### Example 8

The apparatus of any one or more of Examples 6 through 7, wherein the dissection member extends distally from the bulbous portion.

### Example 9

The apparatus of any one or more of Examples 6 through 8, further comprising a second position sensor located in the bulbous portion, wherein the second position sensor is operable to generate signals indicating a position of the distal tip member in space.

### Example 10

The apparatus of Example 9, wherein the second position sensor comprises a second wire coil wrapped about a second coil axis.

### Example 11

The apparatus of Example 10, wherein the second coil axis is laterally offset from the first longitudinal axis.

### Example 12

The apparatus of any one or more of Examples 1 through 11, wherein the dissection member comprises a rigid shaft.

### Example 13

The apparatus of Example 12, wherein the rigid shaft has an atraumatic distal tip.

### Example 14

The apparatus of any one or more of Examples 12 through 13, wherein the rigid shaft extends along a second longitudinal axis, wherein the second longitudinal axis is offset from the first longitudinal axis.

### Example 15

The apparatus of Example 14, wherein the second longitudinal axis is parallel with the first longitudinal axis.

### Example 16

The apparatus of any one or more of Examples 1 through 8 and 12 through 15, further comprising a second position sensor located in the dissection member, wherein the second position sensor is operable to generate signals indicating a position of the dissection member in space.

### Example 17

The apparatus of Example 16, wherein the second position sensor comprises a second wire coil wrapped about a second coil axis.

### Example 18

The apparatus of Example 17, wherein the second coil axis is laterally offset from the first longitudinal axis, wherein the second coil axis is parallel with the first longitudinal axis.

### Example 19

An apparatus comprising: (a) a shaft assembly defining a lumen; (b) a distal tip member at a distal end of the shaft assembly, the distal tip member having an opening in fluid communication with the lumen of the shaft assembly, wherein a longitudinal axis extends centrally through the opening and lumen at the distal tip member; (c) a dissection member extending distally from the distal tip member; (d) a first position sensor operable to generate signals indicating a position of the distal tip member in space, wherein the first position sensor is located proximal to the dissection member, wherein the first position sensor is coaxially positioned about the longitudinal axis; and (e) a second position sensor operable to generate signals indicating a position of the dissection member in space, wherein the second position sensor is longitudinally offset from the first position sensor.

### Example 20

The apparatus of Example 19, wherein a second longitudinal axis extends centrally through the dissection member.

### Example 21

The apparatus of Example 20, wherein the second longitudinal axis is offset from the first longitudinal axis.

### Example 22

The apparatus of any one or more of Examples 20 through 21, wherein the second longitudinal axis is parallel with the first longitudinal axis.

### Example 23

The apparatus of any one or more of Examples 20 through 22, wherein the second position sensor is coaxially positioned about the second longitudinal axis.

### Example 24

The apparatus of any one or more of Examples 19 through 23, wherein the second position sensor is located on or in the dissection member.

### Example 25

An apparatus comprising: (a) a shaft assembly defining a lumen; (b) a distal tip member at a distal end of the shaft assembly, the distal tip member having an opening in fluid communication with the lumen of the shaft assembly, wherein a first longitudinal axis extends centrally through the opening and lumen at the distal tip member; (c) a dissection member extending distally from the distal tip member, wherein a second longitudinal axis extends centrally through the dissection member, wherein the second longitudinal axis is offset from the first longitudinal axis, wherein the second longitudinal axis is parallel with the first longitudinal axis; (d) a first position sensor operable to generate signals indicating a position of the distal tip member in space, wherein the first position sensor is located proximal to the dissection member, wherein the first position sensor is coaxially positioned about the first longitudinal axis; and (e) a second position sensor operable to generate signals indicating a position of the dissection member in space, wherein the second position sensor is proximal to the first position sensor, wherein the second position sensor is coaxially positioned about the second longitudinal axis.

### IV. MISCELLANEOUS

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a shaft assembly defining a lumen;
(b) a distal tip member at a distal end of the shaft assembly, the distal tip member having an opening in fluid communication with the lumen of the shaft assembly, wherein a first longitudinal axis extends centrally through the opening and lumen at the distal tip member;
(c) a dissection member extending distally from the distal tip member; and
(d) a first position sensor operable to generate signals indicating a position of the distal tip member in space.

2. The apparatus of claim 1, wherein the first position sensor comprises a first wire coil wrapped about a first coil axis.

3. The apparatus of claim 2, wherein the first coil axis is coaxial with the first longitudinal axis.

4. The apparatus of claim 1, wherein the first position sensor comprises an annular member captured radially between a distal portion of the shaft assembly and a proximal portion of the distal tip member.

5. The apparatus of claim 4, wherein the shaft assembly includes a proximal portion having a first outer diameter and the distal portion having a second outer diameter, wherein the second outer diameter is smaller than the first outer diameter, wherein the annular member is positioned on the distal portion about the second outer diameter.

6. The apparatus of claim 1, wherein the distal tip member has a bulbous portion.

7. The apparatus of claim 6, wherein the distal tip member further includes a cylindraceous portion located proximal to the bulbous portion.

8. The apparatus of claim 6, wherein the dissection member extends distally from the bulbous portion.

9. The apparatus of claim 6, further comprising a second position sensor located in the bulbous portion, wherein the second position sensor is operable to generate signals indicating a position of the distal tip member in space.

10. The apparatus of claim 9, wherein the second position sensor comprises a second wire coil wrapped about a second coil axis.

11. The apparatus of claim 10, wherein the second coil axis is laterally offset from the first longitudinal axis.

12. The apparatus of claim 1, wherein the dissection member comprises a rigid shaft.

13. The apparatus of claim 12, wherein the rigid shaft has an atraumatic distal tip.

14. The apparatus of claim 12, wherein the rigid shaft extends along a second longitudinal axis, wherein the second longitudinal axis is offset from the first longitudinal axis.

15. The apparatus of claim 14, wherein the second longitudinal axis is parallel with the first longitudinal axis.

16. The apparatus of claim 1, further comprising a second position sensor located in the dissection member, wherein the second position sensor is operable to generate signals indicating a position of the dissection member in space.

17. The apparatus of claim 16, wherein the second position sensor comprises a second wire coil wrapped about a second coil axis.

18. The apparatus of claim 17, wherein the second coil axis is laterally offset from the first longitudinal axis, wherein the second coil axis is parallel with the first longitudinal axis.

19. An apparatus comprising:
(a) a shaft assembly defining a lumen;
(b) a distal tip member at a distal end of the shaft assembly, the distal tip member having an opening in fluid communication with the lumen of the shaft assembly, wherein a longitudinal axis extends centrally through the opening and lumen at the distal tip member;
(c) a dissection member extending distally from the distal tip member;
(d) a first position sensor operable to generate signals indicating a position of the distal tip member in space, wherein the first position sensor is located proximal to the dissection member, wherein the first position sensor is coaxially positioned about the longitudinal axis; and
(e) a second position sensor operable to generate signals indicating a position of the dissection member in space, wherein the second position sensor is longitudinally offset from the first position sensor.

20. The apparatus of claim 19, wherein a second longitudinal axis extends centrally through the dissection member.
